(19) United States
(12) Patent Application Publication
(10) Pub. No.: US 2024/0358506 A1
(43) Pub. Date: Oct. 31, 2024

(54) MECHANICAL WAVE SENSOR FOR DETECTING DETACHMENT OF PROSTHETIC VALVE ACTUATOR

(71) Applicant: EDWARDS LIFESCIENCES CORPORATION, Irvine, CA (US)

(72) Inventors: Joseph Mordechai Leichner, Netanya (IL); Anatoly Dvorsky, Haifa (IL)

(21) Appl. No.: 18/769,639

(22) Filed: Jul. 11, 2024

Related U.S. Application Data
(63) Continuation of application No. PCT/US2023/010506, filed on Jan. 10, 2023.
(60) Provisional application No. 63/300,227, filed on Jan. 17, 2022.

(51) Int. Cl. A61F 2/24

(52) U.S. Cl. CPC A61F 2/2418; A61F 2/2436; A61F 2250/0096

(57) ABSTRACT

The present invention relates to systems, assemblies and methods for detecting detachment of a prosthetic valve actuator. In an example, a prosthetic valve actuator detachment detection system comprises at least one actuator, a frame of the prosthetic valve movable by the at least one actuator between a radially compressed configuration and a radially expanded configuration, and at least one mechanical wave sensor.

### CROSS-REFERENCE TO RELATED APPLICATIONS

This application is a continuation of International Application No. PCT/US2023/010506, filed Jan. 10, 2023, which claims benefit of U.S. Provisional Application No. 63/300,227, filed on Jan. 17, 2022, the contents of each of which are herein incorporated by reference in their entirety.

### FIELD

The present invention relates to the field of prosthetic valves, and in particular to systems, assemblies and methods for detecting detachment of a prosthetic valve actuator.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures is performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Mechanically expandable valves are a category of prosthetic valves that rely on a mechanical actuation mechanism for expansion. The actuation mechanism usually includes a plurality of actuation/locking assemblies, releasably connected to respective actuation members of the valve delivery system, controlled via the handle for actuating the assemblies to expand the valve to a desired diameter. The assemblies may optionally lock the valve's position to prevent undesired recompression thereof, and disconnection of the delivery system's actuation member from the valve actuation/locking assemblies, to enable retrieval thereof once the valve is properly positioned at the desired site of implantation.

After implanting a prosthetic valve, such as a mechanically expandable valve, the actuation mechanism is detached from the prosthetic valve and extracted. If the actuation mechanism has not properly detached, the prosthetic valve will be extracted along with the actuation mechanism. What is desired, and not provided by the prior art, is a mechanism and method for checking that the actuation mechanism has properly detached.

### SUMMARY

The present disclosure is directed toward devices, assemblies and methods for detecting detachment of a prosthetic valve actuator. This is provided in some examples by a prosthetic valve actuator detachment detection system, comprising: at least one actuator; a frame of the prosthetic valve movable by the at least one actuator between a radially compressed configuration and a radially expanded configuration; and at least one mechanical wave sensor.

In some examples, the at least one mechanical wave sensor is a sound sensor. In some examples, the at least one mechanical wave sensor is a vibration sensor.

In some examples, the system further comprises a detection module configured to: receive an output of the at least one mechanical wave sensor; based at least in part on the received output, determine whether the at least one actuator has detached from the frame; and based at least in part on the determination, output an indication of whether the at least one actuator has detached from the frame.

In some examples, the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod based at least in part on the at least one actuator, and wherein the determination whether the at least one actuator has detached from the frame comprises a determination whether the at least one actuator has detached from the at least one rod.

In some examples, each of the proximal member and distal member of each of the at least one axially extending post comprises an inner bore, the respective one of the at least one rod extending through the inner bores of the proximal member and distal member.

In some examples, each of the at least one actuator comprises: a pair of elongated members; and a movable sleeve configured to compress the pair of elongated members such that the compressed pair of elongated members are fastened to a respective one of the at least one rod, wherein when the movable sleeve is moved away from the pair of elongated members the pair of elongated members detach from the respective rod, the determination whether the at least one actuator has detached from the at least one rod comprising a determination whether the pair of elongated members have detached from the respective rod.

In some examples, the at least one axially extending post comprises a plurality of axially extending posts and the at least one rod comprises a plurality of rods, wherein the at least one actuator comprises a plurality of actuators, the rotation of each of the plurality of rods based at least in part on a respective one of the plurality of actuators, and wherein the determination whether the at least one actuator has detached from the frame comprises a determination whether all of the plurality of actuators have detached from the frame.

In some examples, the determination whether the actuator has detached from the frame is based at least in part on detecting whether one or more predetermined mechanical wave signals are present in the output of the mechanical wave sensor.

In some examples, the determination whether the plurality of actuators have detached from the frame is based at least in part on detecting a plurality of predetermined mechanical wave signals, wherein the at least one mechanical wave sensor comprises a microphone array, and wherein, based at least in part on the output of the mechanical wave sensor, the detection module is further configured to: identify which of the plurality of actuators have detached from the frame; and output an indication of the identified actuators.

In some examples, the detection module is further configured to: based at least in part on the received output, determine whether the at least one actuator has broken; and based at least in part on the determination, output an indication of whether the at least one actuator has broken.

In some examples, the at least one mechanical wave sensor is attached to the at least one actuator.

In some examples, the at least one mechanical wave sensor is attached to the frame.

In some examples, the system further comprises a delivery shaft, the at least one actuator supported by the delivery shaft, wherein the at least one mechanical wave sensor is attached to the delivery shaft.

In some examples, the system further comprises a nosecone shaft extending through the frame, wherein the at least one mechanical wave sensor is attached to the nosecone shaft.

In some examples, the system further comprises: a handle; a sensor shaft extending from the handle, the mechanical wave sensor secured to the sensor shaft; and a delivery shaft coupled to the handle, the at least one actuator and the sensor shaft supported by the delivery shaft, wherein the at least one mechanical wave sensor extends distally from the delivery shaft.

In some examples, the system further comprises: a handle; a sensor shaft, the at least one mechanical wave sensor secured to the sensor shaft; and a delivery shaft coupled to the handle, the at least one actuator supported by the delivery shaft and the sensor shaft not supported by the delivery shaft.

In some examples, the at least one mechanical wave sensor is attachable to the skin of a patient.

In some examples, the system further comprises a sensor housing, the at least one mechanical wave sensor attached to the sensor housing, wherein the sensor housing is attachable to the skin of the patient.

Certain examples of the present invention may include some, all, or none of the above advantages. Further advantages may be readily apparent to those skilled in the art from the figures, descriptions, and claims included herein. Aspects and examples of the invention are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following examples and aspects thereof are described and illustrated in conjunction with systems, tools and methods which are meant to be exemplary and illustrative, but not limiting in scope. In various examples, one or more of the above-described problems have been reduced or eliminated, while other examples are directed to other advantages or improvements.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A illustrates a high-level perspective view of a first embodiment of a prosthetic valve, according to some examples;

FIG. 1B illustrates a high-level perspective view of a portion of the prosthetic valve of FIG. 1A, according to some examples;

FIG. 1C illustrates a high-level perspective view of a portion of a second embodiment of a prosthetic valve, according to some examples;

FIG. 2A illustrates a high-level side view of the prosthetic valve of FIG. 1A attached to a delivery system, according to some examples;

FIGS. 2B-2G illustrate various portions of the prosthetic valve and delivery system of FIG. 2A, comprising a mechanical wave sensor, according to some examples;

FIGS. 3A-3C illustrate various steps in the detachment of an actuator from the prosthetic valve of FIG. 1A, according to some examples;

FIG. 4 illustrates a high-level perspective view of a mechanical wave sensor secured to a patient's skin, according to some examples; and

FIG. 5 illustrates a high-level perspective view of a case for storing the prosthetic valve of FIG. 1A, according to some examples.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

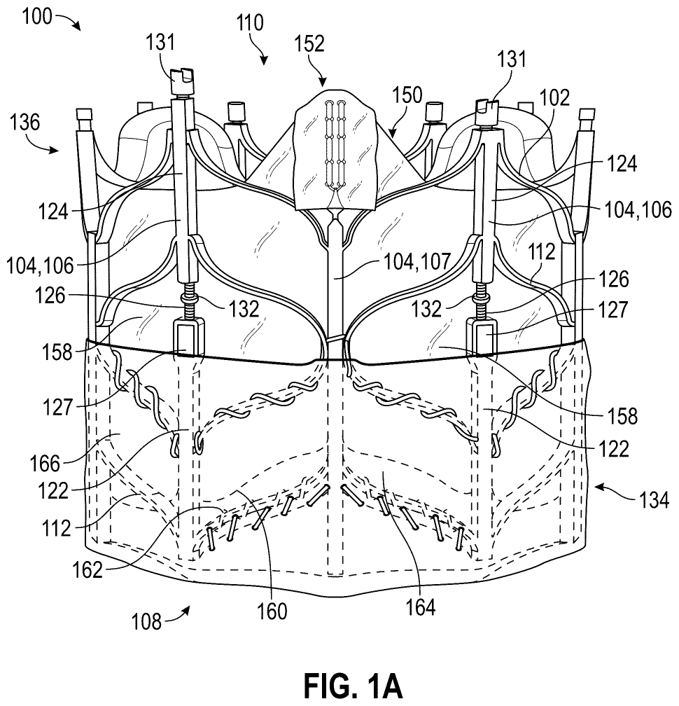

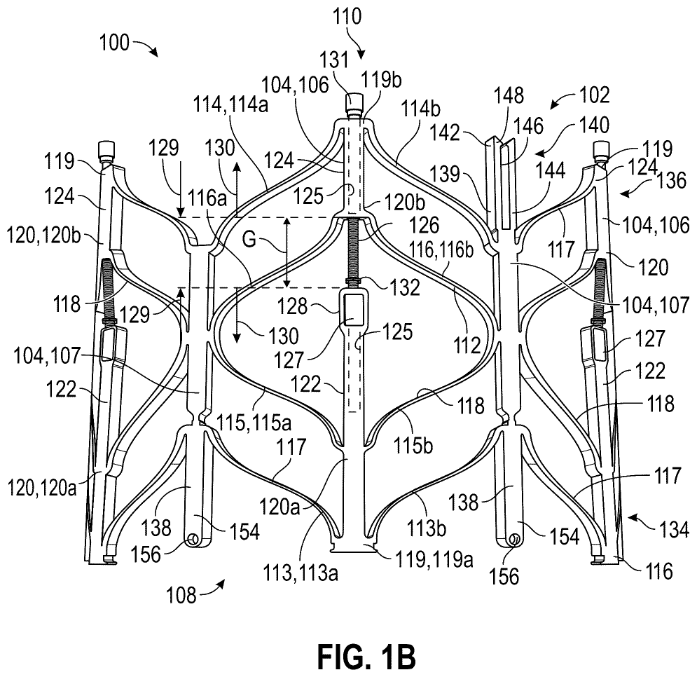

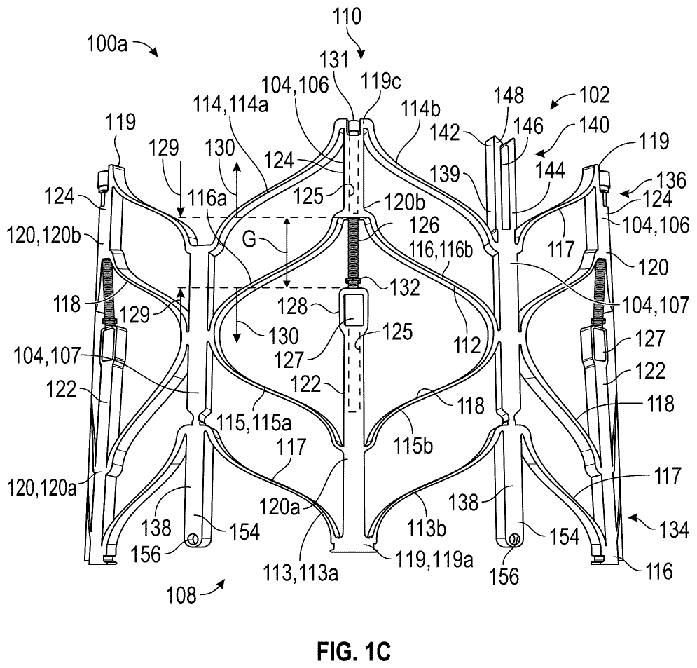

FIG. 1A illustrates a high-level perspective view of a prosthetic valve 100 and FIG. 1B illustrates a high-level perspective view of a portion of prosthetic valve 100, FIGS. 1A-1B being described together. Prosthetic valve 100 comprises a frame 102, a portion of which is illustrated in FIG. 1B. In FIG. 1A, frame 102 is illustrated with an optional valvular structure (e.g., leaflets) and an optional skirt assembly, as will be described below. While only one side of frame 102 is depicted in FIG. 1B, it should be appreciated that frame 102 forms an annular structure having an opposite side that is substantially identical to the portion shown in FIG. 1B, as illustrated in FIG. 1A.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 100 is crimped or retained by a delivery apparatus in a compressed state during delivery, and then expanded to the expanded state once prosthetic valve 100 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

Frame 102 comprises a plurality of axially extending posts 104. In some examples, one or more posts 104 are configured as radial adjustment mechanisms 106. In some examples, one or more posts 104 are configured as support posts 107. In some examples, one or more posts 104 are configured as radial adjustment mechanism 106 and one or more posts 104 are configured as support posts 107. In some examples, radial adjustment mechanisms 106 are integrated into frame 102, thereby reducing the crimp profile and/or bulk of prosthetic valve 100. Integrating radial adjustment mechanisms 106 into frame 102 also simplifies the design of prosthetic valve 100, making the prosthetic valve 100 cheaper and/or easier to manufacture. Radial adjustment mechanism 106 is used to radially expand and/or radially compress frame 102, as will be described below.

Posts 104 extend axially between a distal end 108 and a proximal end 110 of frame 102. In some examples, such as the example shown in FIGS. 1A-1B, distal end 108 is an inflow end and proximal end 110 is an outflow end, such as when prosthetic valve 100 is configured to replace a native aortic valve and prosthetic valve 100 is delivered to the native aortic valve via a retrograde, transfemoral delivery approach (e.g., through a femoral artery and the aorta). However, in other examples, distal end 108 is the outflow end and proximal end 110 is the inflow end, such as when prosthetic valve 100 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

Posts 104 are coupled together by a plurality of circumferentially extending link members or struts 112. Each strut 112 extends circumferentially between adjacent posts 104 to connect all of the axially extending posts 104. In some examples, frame 102 comprises an equal number of support posts 107 and radial adjustment mechanisms 106. In one further example, radial adjustment mechanisms 106 and support posts 107 are arranged in an alternating order such that each strut 112 is positioned between a radial adjustment mechanism and a support post 107, i.e., each strut 112 is coupled on one end to a post 104 that is configured as a radial adjustment mechanism 106 and is coupled on the other end to a post 104 that is configured as a support post 107. However, this is not meant to be limiting in any way, and frame 102 can include different numbers of support posts 107 and radial adjustment mechanisms 106, without exceeding the scope of the disclosure. Similarly, radial adjustment mechanisms 106 and support posts 107 can be arranged in a non-alternating order, without exceeding the scope of the disclosure.

In some examples, as illustrated in FIG. 1B, struts 112 include: a first row of struts 113 at or near distal end 108 of frame 102; a second row of struts 114 at or near proximal end 110 of frame 102; and third and fourth rows of struts 115, 116, respectively, positioned axially between the first and second rows of struts 113, 114. In some examples, struts 112 define a plurality of cells (i.e., openings) in frame 102. For example, struts 113, 114, 115, and 116 at least partially form define a plurality of first cells 117 and a plurality of second cells 118. Specifically, each first cell 117 is formed by two struts 113a, 113b of the first row of struts 113, two struts 114a, 114b of the second row of struts 114, and two of the posts 104. Each second cell 118 is formed by two struts 115a, 115b of the third row of struts 115 and two struts 116a, 116b of the fourth row of struts 116. As illustrated in FIG. 1B, each second cell 118 is disposed within one of first cells 117.

In some examples, as illustrated in FIG. 1B, struts 112 of frame 102 comprise a curved shape. In some examples, each first cell 117 has an axially-extending elliptical shape including first and second apices 119 (e.g., proximal apex 119a and distal apex 119b) disposed at the major vertices of the ellipse. In some examples, each second cell 118 has a circumferentially-extending elliptical shape including first and second apices 120 (e.g., proximal apex 120a and distal apex 120b) disposed at the minor vertices of the ellipse. In some examples, frame 102 comprises six first cells 117 arranged circumferentially in a row, six second cells 118 arranged circumferentially in a row within the six first cells 117, and thirteen posts 104. However, this is not meant to be limiting in any way, and frame 102 can comprise a greater or fewer number of first cells 117 and a correspondingly greater or fewer number of second cells 118 and posts 104, without exceeding the scope of the disclosure.

In some examples, some of posts 104, such as the posts 104 that are configured as radial adjustment mechanisms 106, are discontinuous and each include a distal member 122 and a proximal member 124 that are axially separated from one another by a gap G (FIG. 1B). Proximal member 124 (i.e., the upper post shown in FIG. 1B) extends axially from proximal end 110 of frame 102 towards the respective distal member 122, and distal member 122 (i.e., the lower post shown in FIG. 1B) extends axially from distal end 108 of frame 102 towards the respective proximal member 124. In some examples, each proximal member 124 is axially aligned with a corresponding distal member 122 and one or both of the members 122, 124 include an inner bore 125, so that, for example, the axially aligned pair of members 122, 124 can receive an actuation member, such as in the form of a substantially straight threaded rod 126 as shown in the illustrated example.

Threaded rod 126 is coupled to the respective proximal member 124 and/or distal member 122. In some examples, rod 126 is rotatably coupled to proximal member 124 such that rod 126 can only be rotated to move rod 126 axially relative to proximal member 124 but otherwise cannot move relative to proximal member 124 (e.g., it cannot slide axially relative to proximal member 124 or move circumferentially and/or radially relative to proximal member 124). Similarly, in some examples, rod 126 is restrained radially and/or circumferentially by distal member 122 such that rod 126 can only be rotated and/or moved axially relative to distal member 122 but otherwise cannot move relative to the distal member 122 (e.g., radially and/or circumferentially). In some examples, threaded rod 126 is inserted through an inner bore 125 of one of the proximal members 124 and into a stationary nut 127 and/or bore 125 included in the respective distal member 122. In other examples, threaded rod 126 does extend through a bore in proximal member 124 and instead is secured to proximal member 124 using other suitable structures such as guides, straps, loops, collars, etc.

In some examples, stationary nut 127 is included at a proximal end portion 128 of distal member 122. In some examples, threaded rod 126 extends distally past nut 127 into inner bore 125 of distal member 122. Stationary nut 127 is held in a fixed position relative to distal member 122 such that nut 127 does not rotate relative to distal member 122. In this way, whenever threaded rod 126 is rotated (e.g., by a physician), threaded rod 126 rotates relative to both stationary nut 127 and distal member 122. In some examples, nut 127 includes a threaded bore that that is configured to engage the threads of threaded rod 126 to prevent rod 126 from moving axially relative to nut 127 and distal member 122 unless threaded rod 126 is being rotated. In other examples, in lieu of using nut 127, at least a portion of inner bore 125 of distal member 122 is threaded. For example, proximal end portion 128 of distal member 122 can comprise inner threads configured to engage threaded rod 126 such that rotation of threaded rod 126 causes it to move axially.

When threaded rod 126 is inserted into and/or otherwise coupled to a pair of axially aligned members 122, 124, the pair of axially aligned members served to expand and/or compress frame 102 when threaded rod 126 is rotated, as described below. In some examples, respective threaded rods 126 are inserted through each pair of axially aligned members 122, 124 so that all of the members 122, 124 serve to expand and/or compress frame 102. In some examples, frame 102 comprises six pairs of members 122, 124, and each of the six pairs of members 122, 124 is has a respective threaded rod 126, such that the expansion and/or compression of frame 102 is controlled at six different points. In other examples (not shown), not all pairs of members 122, 124 have a respective threaded rod 126.

The threaded rod 126 is rotated relative to the respective nut 127 and/or the respective member 122 to axially foreshorten and/or axially elongate frame 102, thereby radially expanding and/or radially compressing, respectively, frame 102. Specifically, when threaded rod 126 is rotated relative to nut 127 and/or member 122, the respective members 122, 124 can move axially relative to one another, thereby widening or narrowing the gap G (FIG. 1B) separating members 122 and 124, and thus radially compressing or radially expanding the prosthetic valve 100, respectively. Thus, the gap G (FIG. 1B) between members 122 and 124 narrows as frame 102 is radially expanded and widens as frame 102 is radially compressed. Rotation of threaded rod 126 in a first direction (e.g., clockwise) causes corresponding axial movement of members 122 and 124 toward one another (as shown by arrows 129 in FIG. 1B), thereby radially expanding frame 102, while rotation of the threaded rod 126 in an opposite second direction (e.g., counterclockwise) causes corresponding axial movement of members 122 and 124 away from one another (as shown by arrows 130 in FIG. 1B), thereby radially compressing frame 102.

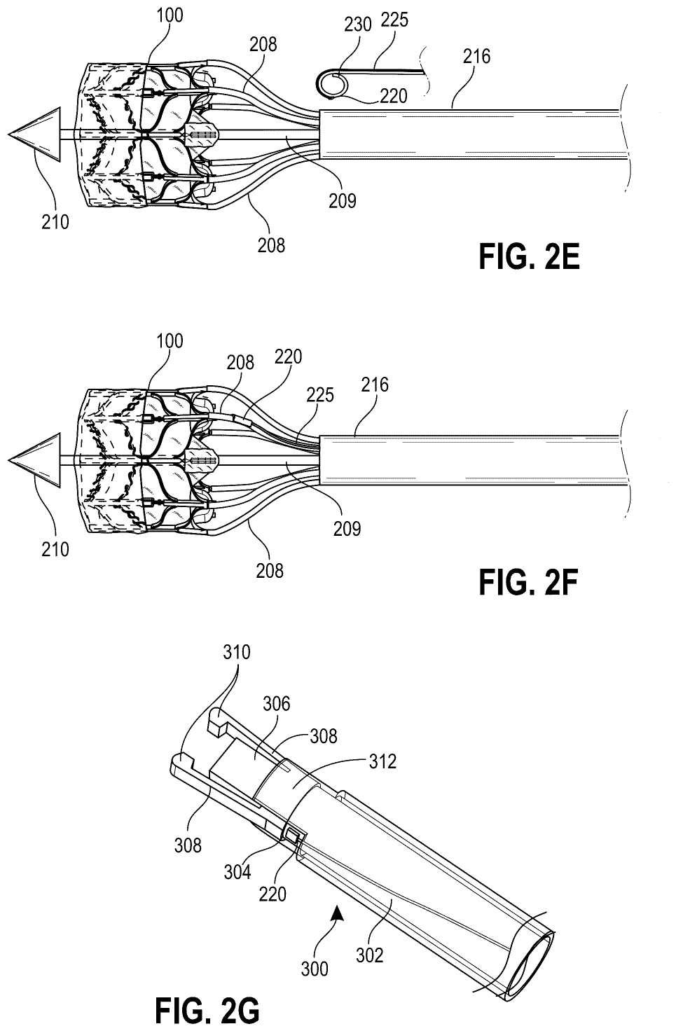

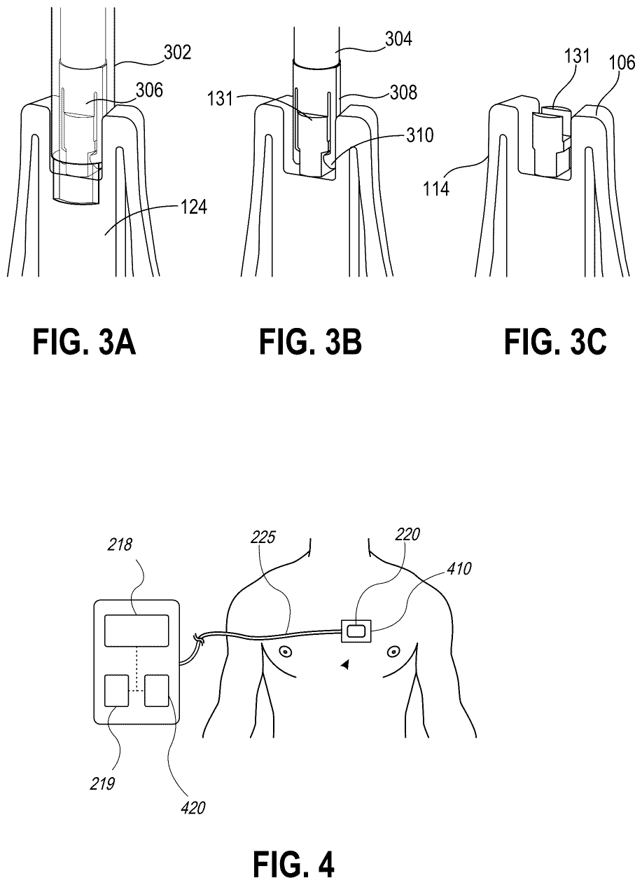

In some examples, threaded rod 126 extends proximally past the proximal end of proximal member 124 and in one further example includes a head portion 131 at its proximal end. Head portion 131 serves at least two functions. First, as will be described in greater detail below with reference to FIGS. 3-4, head portion 131 couples threaded rod 126 to a respective actuator that is used to radially expand and/or radially compress frame 102. Second, head portion 131 prevents proximal member 124 from moving proximally relative to threaded rod 126 and can apply a distally directed force to proximal member 124, such as when radially expanding frame 102. Specifically, in some examples, head portion 131 has a width greater than a diameter of inner bore 125 of the respective proximal member 124 such that head portion 131 is prevented from moving into inner bore 125. Thus, as threaded rod 126 is threaded farther into nut 127, head portion 131 draws closer to nut 127 and distal member 122, thereby drawing proximal member 124 towards distal member 122, and thereby axially foreshortening and radially expanding frame 102.

In some examples, each threaded rod 126 comprises a stopper 132 (e.g., a nut) disposed thereon. Each stopper 132 is disposed on the respective threaded rod 126 such that it sits within gap G. Furthermore, stopper 132 is integrally formed on or fixedly coupled to threaded rod 126 such that it does not move relative to threaded rod 126. Thus, stopper 132 remains in a fixed position on the threaded rod 126 such that it moves together with threaded rod 126. Stopper 132 thus limits the distance of movement for threaded rod 126 in each direction to predetermined values.

As described above, in some examples, a subset of posts 104 are configured as support posts 107. In some examples, support posts 107 extend axially between distal and proximal end portions 134, 136 of frame 102. In some examples, some, or all of support posts 107 extend between a distal end portion 138 and a proximal end portion 139. Proximal end portion 139 of one or more support posts 107 include a commissure support member 140. In some examples, commissure support member 140 comprises first and second commissure arms 142, 144 defining a commissure opening 146 therebetween. In some examples, proximal end of each commissure arm 142, 144 comprises a tooth 148 extending into commissure opening 146.

In some examples, commissure opening 146 extends through a thickness of the respective post 107 and is configured to accept a portion of a valvular structure 150 (e.g., a commissure 152) to couple the valvular structure 150 to frame 102. For example, each commissure 152 is mounted to a respective commissure support member 140, such as by inserting a pair of commissure tabs of adjacent leaflets through opening 146 and suturing the commissure tabs to each other and/or to arms 142, 144. In some examples, opening 146 is fully enclosed by post 107 (e.g., not extending to the proximal edge) such that a portion of valvular structure 150 is slid radially (rather than axially) into the commissure opening 146. Teeth 148 can help retain commissure 152 within commissure opening 146. In the illustrated example, commissure opening 146 has a substantially rectangular shape and extends to the distal end of the respective post 107, however this is not meant to be limiting in any way. In other examples, the commissure opening can have any of a variety of shapes (e.g., square, oval, square-oval, triangular, L-shaped, T-shaped, C-shaped, etc.), without exceeding the scope of the disclosure.

Though only one support post 107 comprising a commissure support member 140 is shown in FIG. 1B, it should be noted that frame 102 can comprise any number of support posts 107, any number of which comprising commissure support members 140. For example, frame 102 can comprise six support posts 107, three of which comprising commissure support members 140, as illustrated. However, in other examples, frame 102 can comprise more or less than six support posts 107 and/or more or less than three commissure support members 140.

In some examples, distal end portion 138 of each support post 107 comprises an extension 154 that extends toward the distal end 108 of frame 102. In some examples, extension 154 comprises an aperture 156 extending radially through a thickness of the respective extension 154. In some examples, extension 154 extends such that a distal edge of extension 154 aligns with or substantially aligns with distal end 108 of the frame 102. During use, extension 154 can prevent or mitigate portions of an outer skirt from extending radially inwardly and thereby prevent or mitigate obstruction of flow through distal end 108 of frame 102 caused by the outer skirt. Extensions 154 can further serve as supports to which portions of the inner and/or outer skirts is coupled. For example, sutures used to connect the inner and/or outer skirts is wrapped around the extensions 154 and/or can extend through openings 156.

In some examples, frame 102 is a unitary and/or fastener-free frame that is constructed from a single piece of material (e.g., Nitinol). In some examples, the plurality of cells 117, 118 are formed by removing portions (e.g., via laser cutting) of the single piece of material. Threaded rods 126 can then be inserted through the bores 125 in proximal members 124 and threaded into threaded nuts 127.

As illustrated in FIG. 1A, prosthetic valve 100 further comprises valvular structure 150, which is coupled to and supported inside frame 102. Valvular structure 150 is configured to regulate the flow of blood through prosthetic valve 100, from the inflow end to the outflow end. In some examples, valvular structure 150 includes a leaflet assembly comprising one or more leaflets 158 made of flexible material. Leaflets 158 are made from in whole or part, biological material, bio-compatible synthetic materials, or other such materials. Suitable biological material can include, for example, bovine pericardium (or pericardium from other sources). Leaflets 158 are secured to one another at their adjacent sides to form commissures 152, each of which is secured to a respective post 104 (e.g., to a support post 107) and/or to other portions of frame 102.

In the example illustrated in FIG. 1A, valvular structure 150 includes three leaflets 158, which are arranged to collapse in a tricuspid arrangement. Each leaflet 158 has a proximal edge portion 160. As shown in FIG. 1A, in some examples, proximal edge portions 160 of leaflets 158 define an undulating, curved scallop edge that follows or tracks portions of struts 112 of frame 102 in a circumferential direction when frame 102 is in a radially expanded configuration. The proximal edges 160 of leaflets is referred to as a “scallop line.”

In some examples, as shown in FIG. 1A, proximal edge portions 160 of leaflets 158 are sutured to an inner skirt 164 generally along the scallop line. Inner skirt 164 can in turn be sutured, via one or more sutures 162, to adjacent struts 112 of frame 102. In other examples, leaflets 158 are sutured directly to frame 102 along the scallop line. Inner skirt 164 can function as a sealing member to prevent or decrease perivalvular leakage, to anchor leaflets 158 to frame 102, and/or to protect leaflets 158 against damage caused by contact with frame 102 during crimping and during working cycles of prosthetic valve 100.

In some examples, prosthetic valve 100 further comprises an outer skirt 166 mounted on an outer surface of frame 102. Outer skirt 166 can function as a sealing member for prosthetic valve 100 by sealing against the tissue of the native valve annulus and helping to reduce paravalvular leakage past prosthetic valve 100. Inner and outer skirts 164, 166 are formed from any of various suitable biocompatible materials, including any of various synthetic materials, including fabrics (e.g., polyethylene terephthalate fabric) or natural tissue (e.g., pericardial tissue).

FIG. 1C illustrates a portion of a prosthetic valve 100a. Prosthetic valve 100a is in all respects similar to prosthetic valve 100, with the exception that struts 114a and 114b extend proximally along head portions 131 into apex 119c.

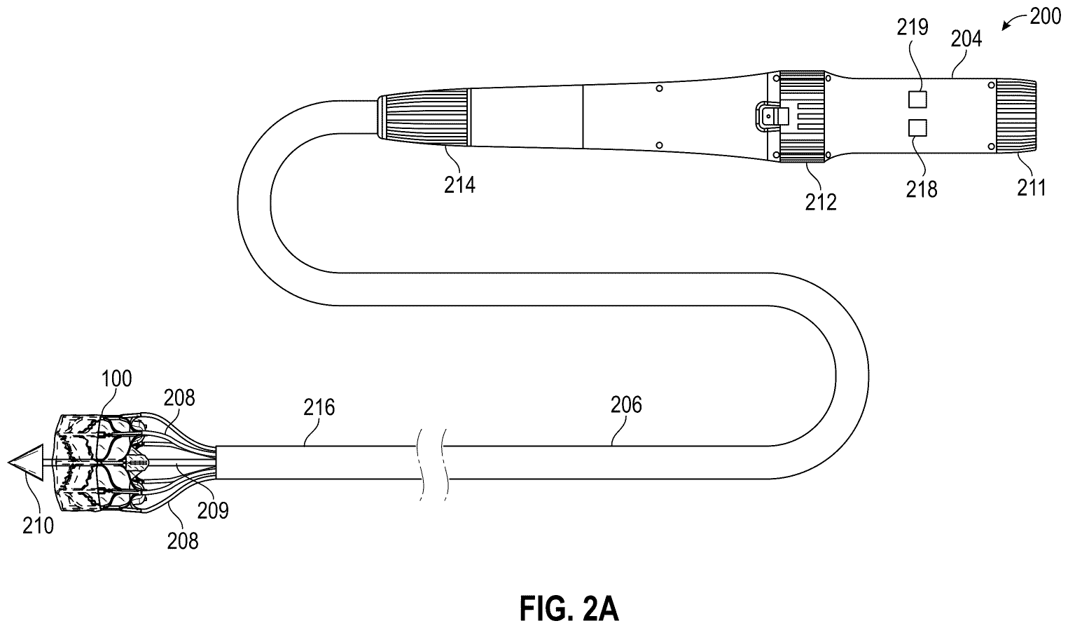

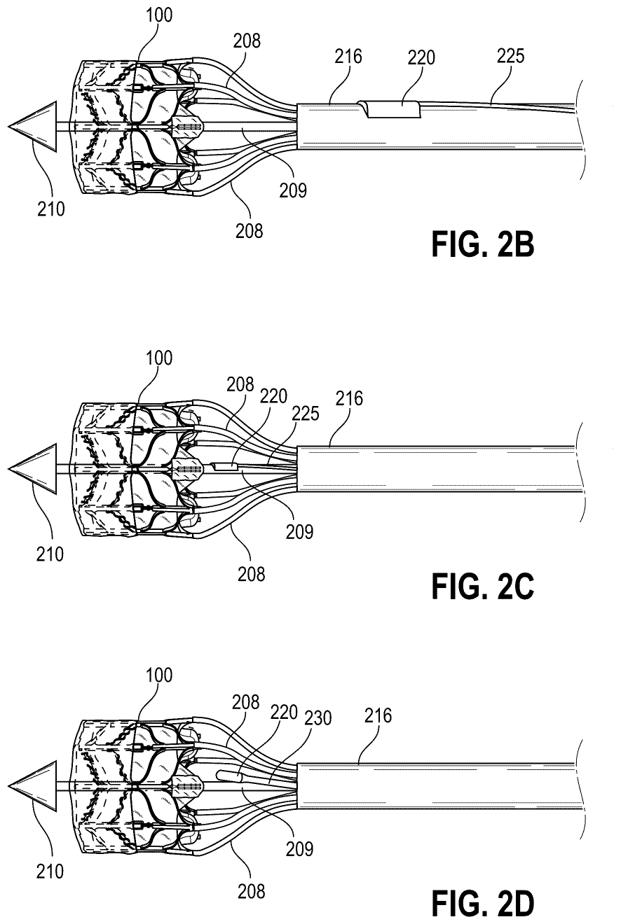

As described above, threaded rods 126 are removably coupled to an actuator assembly of a delivery apparatus. FIG. 2A illustrates a high-level side view of prosthetic valve 100 secured to a delivery apparatus 200 for delivering prosthetic valve 100 to a desired implantation location. It should be understood that delivery apparatus 200 and other delivery apparatuses disclosed herein can be used to implant prosthetic devices other than prosthetic valves, such as stents or grafts.

In the illustrated example, delivery apparatus 200 comprises: a handle 204; a first elongated shaft 206 (which comprises an delivery shaft in the illustrated example) extending distally from the handle 204; at least one actuator 208 extending distally through the delivery shaft 206; an elongated nosecone shaft 209 (which comprises an inner shaft in the illustrated example) extending through first shaft 206; and a nosecone 210 coupled to a distal end portion of nosecone shaft 209. In some examples, nosecone shaft 209 and nosecone 210 define a guidewire lumen for advancing prosthetic valve 100 through a patient's vasculature over a guidewire. Actuator 208 is configured to radially expand and/or radially collapse frame 102 when actuated, such as by one or more knobs 211, 212 and 214 included on handle 204 of delivery apparatus 200. Though the illustrated example shows six actuators 208, this is not meant to be limiting in any way. In some examples, a respective actuator 208 is provided for each threaded rod 126 provided in frame 102.

In some examples, a distal end portion 216 of shaft 206 is sized to house prosthetic valve 100 in its radially compressed, delivery state during delivery thereof through the patient's vasculature. In this manner, distal end portion 216 of shaft 206 functions as a delivery sheath or capsule for prosthetic valve 100 during delivery.

The term “proximal”, as used herein, generally refers to the side or end of any device or a component of a device, which is closer to handle 204 or an operator of handle 204 when in use.

The term “distal”, as used herein, generally refers to the side or end of any device or a component of a device, which is farther from handle 204 or an operator of handle 204 when in use.

As will be described below, actuators 208 are releasably coupled to frame 102. Particularly, when actuated, actuator 208 transmits pushing and/or pulling forces from handle 204 to portions of frame 102 to radially expand and collapse the prosthetic valve as previously described. In some examples, actuators 208 are at least partially disposed within, and extend axially through, one or more lumens of delivery shaft 206. For example, actuators 208 can extend through a central lumen of the shaft 206 or through separate respective lumens formed in the shaft 206.

In some examples, the first knob 211 is a rotatable knob configured to produce axial movement of delivery shaft 206 relative to prosthetic valve 100 in the distal and/or proximal directions in order to deploy prosthetic valve 100 from delivery shaft 206 once prosthetic valve 100 has been advanced to a location at or adjacent to the desired implantation location with the patient's body. For example, rotation of the first knob 211 in a first direction (e.g., clockwise) retracts distal portion 216 of delivery shaft 206 proximally relative to prosthetic valve 100 and rotation of the first knob 211 in a second direction (e.g., counter-clockwise) advances distal portion 216 distally. In other examples, the first knob 211 can be actuated by sliding or moving knob 211 axially, such as pulling and/or pushing the knob. In other examples, actuation of first knob 211 (rotation or sliding movement of the knob 211) produce axial movement of actuators 208 (and therefore prosthetic valve 100) relative to distal portion 216 of delivery shaft 206 to distally advance prosthetic valve 100.

In some examples, second knob 212 is a rotatable knob configured to produce radial expansion and/or compression of frame 102. For example, rotation of second knob 212 can rotate threaded rods 126 of frame 102 via actuators 208, as will be described below. Rotation of second knob 212 in a first direction (e.g., clockwise) radially expands frame 102 and rotation of the second knob 212 in a second direction (e.g., counter-clockwise) radially collapses frame 102. In other examples, second knob 212 is actuated by sliding or moving knob 212 axially, such as pulling and/or pushing the knob.

In some examples, third knob 214 is a rotatable knob operatively coupled to a proximal end portion of each actuator 208. In some examples, third knob 214 is configured to retract an outer sleeve or support tube of each actuator 208 to disconnect actuators 208 from threaded rods 126 of frame 102, as further described below. Once actuators 208 are uncoupled from frame 102, delivery apparatus 200 can be removed from the patient, leaving just prosthetic valve 100 in the patient.

In some examples, delivery apparatus 200 and/or prosthetic valve 100 further comprise: a detection module 218; an indicator 219; at least one mechanical wave sensor 220; and a communication line 225. In some examples, communication line 225 connects at least one mechanical wave sensor 220 to detection module 218, however this is not meant to be limiting in any way. In some examples (not shown), a communication component is further provided for communication with external systems.

In some examples (not shown), a memory is further provided and is configured to store signals received from mechanical wave sensor 220. A memory may include a suitable memory chip or storage medium such as, for example, a PROM, EPROM, EEPROM, ROM, flash memory, solid state memory, or the like.

In some examples, communication line 225 comprises various electrically conductive materials, such as copper, aluminum, silver, gold, and various alloys such as tentalum/platinum, MP35N and the like. An insulator (not shown) can surround communication line 225. The insulator can include various electrically insulating materials, such as electrically insulating polymers.

In some examples, communication line 225 is not provided, and at least one mechanical wave sensor 220 is in wireless communication with detection module 218. In some examples, the wireless communication can include a radio frequency (RF) link, an infrared link, a Bluetooth link, or any other known wireless communication methods.

In some examples, detection module 218 is implemented as one or more of: a dedicated circuitry; a micro-controller; a processor; an application-specific integrated circuit (ASIC); a field-programmable-gate-array (FPGA); or software running on a computing device. In an example where detection module 218 is implemented as software, a memory (not shown) is further provided, the memory having stored therein instructions which when run by one or more processors cause the one or more processors to run the software. In some examples, indicator 219 comprises a display.

The term “mechanical wave sensor”, as used herein, means a sensor that senses mechanical waves. In some examples, at least one mechanical wave sensor 220 is a sound sensor. The term “sound sensor”, as used herein, means a sensor that senses acoustic sounds. In an example where at least one mechanical wave sensor 220 is a sound sensor, mechanical wave sensor is configured to generate electric signals representative of sounds sensed thereby.

According to some examples, at least one mechanical wave sensor 220 comprises a microphone. In some examples, the microphone is any of a piezoelectric, a piezoresistive, or a capacitive-type microphone. A piezoelectric microphone may be made from any piezoelectric material, including piezocomposites, piezoceramics, piezoplastics and the like. In some examples, at least one mechanical wave sensor 220 comprises a piezoelectric film, such as polyvinylidine fluoride (PVDF), which takes the form of a thin plastic polymer sheet and may have a thin electrically conductive nickel copper alloy deposited on each side. At least one mechanical wave sensor 220 may act as a strain gage that generates an electrical signal when a diaphragm included therein vibrates in response to sounds. In some examples, at least one mechanical wave sensor 220 comprises a micro-electrical mechanical system (MEMS) microphone. Although at least one mechanical wave sensor 220 is described as having a single sound sensor, this is not meant to be limiting in any way. In some examples, at least one mechanical wave sensor 220 comprises a plurality of microphones, such as a dedicated microphone array.

In some examples, at least one mechanical wave sensor 220 is a vibration sensor. The term “vibration sensor, as used herein, means a sensor that senses vibrations. In some examples, the vibration sensor is an accelerometer, or any other type of motion sensor that is configured to detect vibrations. In an example where at least one mechanical wave sensor 220 is a vibration sensor, mechanical wave sensor is configured to generate electric signals representative of vibrations sensed thereby.

In some examples, at least one mechanical wave sensor 220 is provided within a housing. In some examples, at least one mechanical wave sensor 220, and/or the provided housing, is formed of biocompatible material. The biocompatible material is a biocompatible polymer material selected from but not limited to, poly(dimethyl siloxane) (PDMS), polycaprolactone (PCL), methyl-vinyl siloxane, ethylene/vinyl acetate copolymers, polyethylene, polypropylene, ethylene/propylene copolymers, acrylic acid polymers, ethylene/ethyl acrylate copolymers, polytetrafluoroethylene (PTFE), polyurethanes, thermoplastic polyurethanes and polyurethane elastomers, polybutadiene, polyisoprene, poly(methacrylate), polymethyl methacrylate, styrene-butadiene-styrene block copolymers, poly(hydroxyethyl-methacrylate) (pHEMA), polyvinyl chloride, polyvinyl acetate, polyethers, polyacrylo-nitriles, polyethylene glycol (PEG), polymethylpentene, polybutadiene, polyhydroxy alkanoates, poly(lactic acid) (PLA), poly(glycolic acid) (PGA), polyanhydrides, polyorthoesters, and copolymers and combinations thereof.

In some examples, at least one mechanical wave sensor 220 is attached to a component of delivery apparatus 200 and/or prosthetic valve 100 such that the distance between at least one mechanical wave sensor 220 and head portions 131 of threaded rods 126, during valve expansion and/or compression, is not greater than 20 centimeters. In some examples, at least one mechanical wave sensor 220 is attached to a component of delivery apparatus 200 and/or prosthetic valve 100 such that the distance between at least one mechanical wave sensor 220 and head portions 131 of threaded rods 126, during valve expansion and/or compression, is not greater than 10 centimeters. In some examples, at least one mechanical wave sensor 220 is attached to a component of delivery apparatus 200 and/or prosthetic valve 100 such that the distance between at least one mechanical wave sensor 220 and head portions 131 of threaded rods 126, during valve expansion and/or compression, is not greater than 5 centimeters.

If at least one mechanical wave sensor 220 is attached to a component that may move relative to head portions 131 of threaded rods 126 during valve expansion and/or compression, in some examples the distance between mechanical wave sensor and head portions 131 may change during valve expansion and/or compression. In such cases, the distance should optionally not exceed the above-mentioned values in a state of maximal valve expansion and/or compression, i.e., when prosthetic valve 100 is expanded to a maximal desired expansion diameter thereof and/or compressed to a minimum diameter thereof.

It is to be understood that the distance between at least one mechanical wave sensor 220 and head portions 131 of threaded rods 126 is measured between a central point of at least one mechanical wave sensor 220 and the furthest head portion 131 therefrom.

In some examples, as illustrated in FIG. 2B, at least one mechanical wave sensor 220 is attached to distal end portion 216 of delivery shaft 206. In some examples, as illustrated, at least one mechanical wave sensor 220 is attached to the outer surface of delivery shaft 206. In some examples, mechanical wave sensor is attached to the inner surface of delivery shaft 206.

In some examples, as illustrated in FIG. 2C, at least one mechanical wave sensor 220 is attached to nosecone shaft 209. Preferably, at least one mechanical wave sensor 220 is attached to a portion of nosecone shaft 209 that is in the vicinity of head portions 131 of threaded rods 126, as described above. As shown in FIG. 2C, at least one mechanical wave sensor 220 is attached to an outer surface of nosecone shaft 209 at a position that is proximal to prosthetic valve 100 during valve expansion, however this is not meant to be limiting in any way. In some examples, at least one mechanical wave sensor 220 is attached to nosecone shaft 209 at a position that is either within the internal lumen defined by prosthetic valve 100, or distal to prosthetic valve 100, during valve expansion. In some examples (not shown), at least one mechanical wave sensor 220 is attached to nosecone 210.

In some examples, as illustrated in FIG. 2D, delivery apparatus 200 further comprises a sensor shaft 230 extending from handle 204 through delivery shaft 206, and at least one mechanical wave sensor 220 is attached to a distal end of sensor shaft 230. In some examples, as illustrated, at least one mechanical wave sensor 220 extends distally from distal portion 216 of delivery shaft 206. In some examples, sensor shaft 230 is axially movable relative to distal portion 216 of delivery shaft 206.

In some examples, as illustrated in FIG. 2E, sensor shaft 230 is provided independently and externally to delivery shaft 206. In such an example, sensor shaft 230 is optionally axially movable relative to any component of delivery apparatus 200 and prosthetic valve 100. In some examples, sensor shaft 230 is provided in the form of a pigtail catheter, as illustrated in FIG. 2E.

In some examples (not shown), at least one mechanical wave sensor 220 is attached to any other shaft of delivery apparatus 200.

In some examples, as illustrated in FIG. 2F, at least one mechanical wave sensor 220 is attached to an actuator 208. In some examples, as illustrated in FIG. 2F, at least one mechanical wave sensor 220 is attached to an outer surface of actuator 208, however this is not meant to be limiting in any way. In some examples (not shown), a plurality of mechanical wave sensors 220 are provided, each mechanical wave sensor 220 attached to a respective one of a plurality of actuators 208.

FIG. 2G illustrates a high-level perspective view of a distal end 300 of an actuator 208. In some examples, distal end 300 of actuator 208 comprises: a sleeve 302; a driver 304; a central protrusion 306; and a pair of elongated members 308, each comprising a protrusion, or tooth, at a distal end 310 thereof. In some examples, central protrusion 306 extends distally from driver 304. In some examples, elongated members 308 extend distally from the sides of central protrusion 306, such that distal ends 310 are positioned distally to central protrusion 306. In some examples, as illustrated in FIG. 2G, mechanical wave sensor 220 is attached to driver 304, however this is not meant to be limiting in any way. Sleeve 302 surrounds driver 304, and is arranged to be axially moved over elongated members 308, as will be described below.

FIGS. 3A-3C illustrate various steps in the detachment of an actuator 208 from prosthetic valve 100a. Although the following is described in relation to prosthetic valve 100a, this is not meant to be limiting in any way, and the described steps can also be performed for prosthetic valve 100. A proximal portion of a radial adjustment mechanism 106 is illustrated. As illustrated in FIG. 3A, central protrusion 306 is situated within a slit of head portion 131 and distal ends 310 of elongated members 308 are secured distally to head portion 131 by sleeve 302. Sleeve 302 is shown transparently for purposes of illustration. As illustrated, sleeve 302 compresses elongated members 308 such that they are secured to head portion 131. Thus, in this position, driver 304 can be rotated, as described above, thereby rotating head portion 131 and expanding/compressing prosthetic valve 100a.

In some examples, central protrusion 306 is sized and shaped such that it is spaced apart from the inner walls of outer sleeve 302, such that the central protrusion 306 does not frictionally contact outer sleeve 302 during rotation. Although in the illustrated example central protrusion 306 has a substantially rectangular shape in its cross-section, this is not meant to be limiting in any way, and protrusion 306 can have any of various shapes, for example, square, triangular, oval, etc, without exceeding the scope of the disclosure.

In some examples, the distal end portion of sleeve 302 comprises a pair of support extensions 312 extending distally therefrom. In one further example, support extensions 312 are oriented such that, when central protrusion is inserted within head portion 131, support extensions 312 extend partially over a proximal end portion (e.g., the upper end portion) of proximal members 124. The engagement of support extensions 312 with proximal member 124 in this manner can counter-act rotational forces applied to the frame by threaded rods 126 during expansion of prosthetic valve 100a. In the absence of a counter-force acting against these rotational forces, the frame can tend to “jerk” or rock in the direction of rotation of the rods when they are actuated to expand the frame. The illustrated configuration is advantageous in that sleeves 302, when engaging proximal members 124 of the frame, can prevent or mitigate such jerking or rocking motion of the frame.

In some examples, sleeve 302 is advanced (moved distally) and/or retracted (moved proximally) relative to driver 304 via a control mechanism (e.g., knob 214) on handle 204 of delivery apparatus 200, by an electric motor, and/or by another suitable actuation mechanism. For example, the physician can turn knob 214 in a first direction to apply a distally directed force to sleeve 302 and can turn knob 214 in an opposite second direction to apply a proximally directed force to sleeve 302. Thus, when sleeve 302 does not abut prosthetic valve 100a and the physician rotates knob 214 in the first direction, sleeve 302 can move distally relative to driver 304, thereby advancing the sleeve 302 over driver 304 and elongated members 308. When sleeve 302 does abut prosthetic valve 100a, the physician can rotate knob 214 in the first direction to push the entire prosthetic valve 100a distally via sleeve 302. Furthermore, when the physician rotates knob 214 in the second direction, sleeve 302 can move proximally relative to driver 304, thereby withdrawing/retracting sleeve 302 from the driver 304.

When expansion of prosthetic valve 100a is completed, sleeve 302 is retracted proximally off of elongated members 308, thus releasing elongated members 308, as described above. Particularly, elongated members 308 are flexible, such that when not compressed by sleeve 302 they bend outwards, away form head portion 131. Actuator 208 is then retracted, as illustrated in FIG. 3C.

Although the above has been described in relation to an example where distal end 300 of actuator 208 comprises a central protrusion 306, which is inserted within a respective slit of head portion 131, this is not meant to be limiting in any way, and actuator 208 can connect to head portion 131 in other suitable configurations, without exceeding the scope of the disclosure.

Detection module 218 receives an output of at least one mechanical wave sensor 220 and based at least in part on the received output, determines whether actuator 208 has detached, i.e. whether elongated members 308 have been released from head portion 131. In some examples, detection module 218 detects one or more predetermined mechanical wave signals within the output of at least one mechanical wave sensor 220 to determine whether actuator 208 has detached. Particularly, in some examples, detection module 218 identifies the sound, or vibration, caused by elongated members 308 being released from the position where they were secured against head portion 131 of threaded rod 126. In some examples, the sound/vibration caused by elongated members 308 being released is identified by identifying a short sound/vibration, differing from the ambient noise. Detection module 218 outputs an indication whether actuator 208 has detached successfully. In some examples, the indication is output at indicator 219. In some examples, the indication, and/or additional data, is output to external systems.

In some examples, mechanical wave sensor 220 has high signal-to-noise ratio, high sensitivity, suitable ambient noise shrouding capability, and/or the ability to measure low frequency signals, in order to overcome various possible disturbances such as background noise or the relatively low amplitude of the vibrations or sounds that are generated by heart activity, lung activity, and/or blood flow.

In some examples (not shown), an ambient noise sensor (not shown) is provided, the ambient noise sensor configured to acquire environment noise and/or speech. The ambient noise sensor is used to remove environmental noise from measured signals. Alternatively, other known filtering algorithms are applied by detection module 218 to remove environmental noise from the receive output of mechanical wave sensor 220. In some examples, the output of mechanical wave sensor 220 is continuously monitored by detection module 218 to identify the repetitive, ambient noises/vibrations. As a result, detection module 218 can detect any noises/vibrations that are not part of the ambient noises/vibrations.

In some examples, detection module 218 further analyzes the output of mechanical wave sensor 220 to detect sounds and/or vibrations generated by the heart. Sounds/vibrations from the heart are distinguished from the release sound/vibration of elongated members 308 due to having, for example, different amplitudes, waveforms and/or frequencies. Heart sound sensors are conventionally utilized for detection of sounds generated by heart activity, which include the opening and closing of the native heart valves and blood flow. The human heart sounds include a first major sound (“S1”) and second major sound (“S2”). The first major sound usually includes a mitral valve sound (“M1”) and a tricuspid valve sound (“T1”), while the second major sound usually includes an aortic valve sound (“A2”) and a pulmonary valve sound (“P2”).

Heart sounds, such as S1 and S2, are repetitive sounds generated by the ongoing cycling heart activity, while sounds caused by the release of elongated members are non-repetitive sounds that occur only during the release thereof from frame 102, and will have different amplitudes, waveforms and/or frequencies than those of heart sounds.

In some examples, heart sounds monitoring is not required during valve expansion, such that the heart sounds are actually part of background sounds that should be filtered out, as described above.

In some examples (not shown), ECG sensors are provided. In such an example, the ECG sensors can be utilized for measuring, potentially in a simultaneous manner, actuator release sounds and ECG signals, with or without heart sounds.

In some examples, where mechanical wave sensor 220 is attached to an outer surface of actuator 208, a lubricious coating is provided on an outer surface of driver 304 and/or the inner surface of sleeve 302. The lubricious coating can include Teflon, parylene, PTFE, polyethylene, polyvinylidene fluoride, and combinations thereof. Suitable materials for a lubricious coating also include other materials desirably having a coefficient of friction of 0.1 or less. Such coating can allow sleeve 302 to be tightly disposed around actuator driver 304 in a manner that will allow vibrations to be transmitted from elongated members 308 to sleeve 302, so as to be easily sensed by a corresponding mechanical wave sensor 220 implemented as a vibration sensor, while allowing driver 304 and sleeve 302 to be conveniently movable relative to each other along the axial axis.

As described above, in some examples a respective mechanical wave sensor 220 is attached to each actuator 208. In such an example, detection module 218 can separately detect whether each actuator 208 has detached from frame 102. Alternatively, in an example where only a single mechanical wave sensor 220 is provided, if each actuator 208 is detached at a different time, detection module can analyze the output of mechanical wave sensor 220 at the time of detachment to determine whether the respective actuator 208 was detached.

In some examples, where a separate mechanical wave sensor 220 is not provided for each actuator, and actuators 208 are detached simultaneously, detection module 218 identifies which actuators 208 were successfully detached in accordance with the distance between mechanical wave sensor 220. Particularly, a sound wave being generated by detachment of a first actuator 208 arrives at mechanical wave sensor 220 at a different time than a respective sound wave generated by detachment of a second actuator 208. Thus, in the example where six actuators 208 are provided, if detection module 218 detects a detachment sound six times, an indication that all actuators 208 have detached is output. If one of the actuators 208 did not detach, detection module 218 can identify which one it is based on the time intervals between the identified successful detachments.

In some examples, detection module 218 is further configured to determine whether one or more actuator 208 has broken. Particularly, upon breakage, an actuator 208 will generate a unique sound, different than the sound generated when elongated members 308 detach from head portion 131. Thus, if the respective predetermined sound associated with breakage is detected, detection module 218 determines that the respective actuator 208 has broken and outputs an indication of the breakage.

FIG. 4 illustrates an example where mechanical wave sensor 220 is attached to the skin of the patient. In such an example, mechanical wave sensor is secured to an extracorporeal sensor housing 410, that is placed in contact with the patient's body. In some examples (not shown), extracorporeal sensor housing 410 is positioned in close proximity to the patient's body. In such an example, mechanical wave sensor 220 is configured to externally measure sounds produced within the body of the subject. The extracorporeal mechanical wave sensor 220 is placed over the patient's chest, as shown in FIG. 4, prior to or during a prosthetic valve implantation procedure.

In some examples, extracorporeal sensor housing 410 is provided in the form of a patch. In some examples, extracorporeal sensor housing 410 is provided in the form of a precordial patch, configured to be placed on or attached to a portion of the patient's body that includes the anterior surface of the lower thorax.

In some examples, detection module 218 and indicator 219 are not positioned within handle 204, and are rather positioned separately near the patient. In one further example, a communication component 420 is further provided, in communication with detection module 218. In some examples, communication component 420 comprises a transmitter, a receiver, a transceiver, and/or a data communication socket.

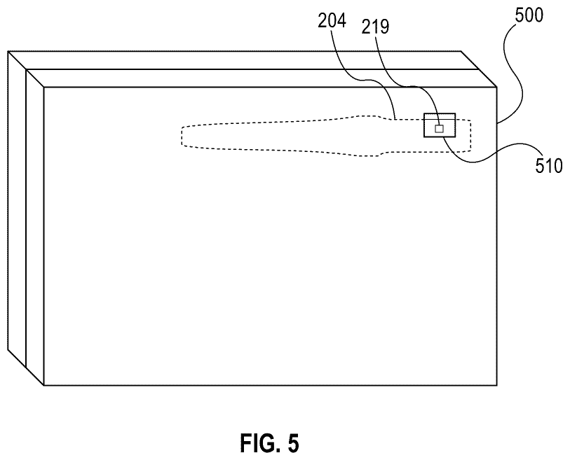

In some examples, indicators on handle 204 can be utilized to determine whether one or more actuators 208 have detached from frame 102 during transport. This can be determined before implantation of the device within a patient and/or at any point during the life cycle of prosthetic valve 100 and delivery apparatus 200. For example, as illustrated in FIG. 5, a case 500 can be provided, which contains prosthetic valve 100 and delivery apparatus 200 (not shown). In some examples, case 500 comprises a window 510. Window 510 is aligned with handle 204 such that indicator 219 can be seen via window 510. Thus, a detachment of one or more actuators 208 can be detected at any point in time.

In some examples (not shown), an indicator is provided on an external surface of case 500. In some examples (not shown), case 500 is transparent, thereby allowing indicator 219 to be seen at any point in time.

### SOME EXAMPLES OF THE DISCLOSED TECHNOLOGY

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve actuator detachment detection system, comprising: at least one actuator; a frame of the prosthetic valve movable by the at least one actuator between a radially compressed configuration and a radially expanded configuration; and at least one mechanical wave sensor.

Example 2. The system of any example herein, particularly example 1, wherein the at least one mechanical wave sensor is a sound sensor.

Example 3. The system of any example herein, particularly example 1, wherein the at least one mechanical wave sensor comprises a plurality of mechanical wave sensors.

Example 4. The system of any example herein, particularly example 3, wherein the plurality of mechanical wave sensors comprises a plurality of sound sensors.

Example 5. The system of any example herein, particularly example 3, wherein the plurality of mechanical wave sensors comprises a plurality of vibration sensors.

Example 6. The system of any example herein, particularly example 3, wherein the plurality of mechanical wave sensors comprises at least one sound sensor and at least one vibration sensor.

Example 7. The system of any example herein, particularly example 1, wherein the at least one mechanical wave sensor is a vibration sensor.

Example 8. The system of any example herein, particularly example 1, wherein the at least one mechanical wave sensor comprises a microphone.

Example 9. The system of any example herein, particularly example 8, wherein the microphone is selected from a piezoelectric, a piezoresistive, and a capacitive-type microphone.

Example 10. The system of any example herein, particularly example 8, wherein the microphone is a MEMS microphone.

Example 11. The system of any example herein, particularly any one of examples 1 to 10, further comprising a detection module configured to: receive an output of the at least one mechanical wave sensor; based at least in part on the received output, determine whether the at least one actuator has detached from the frame; and based at least in part on the determination, output an indication of whether the at least one actuator has detached from the frame.

Example 12. The system of any example herein, particularly example 11, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod based at least in part on the at least one actuator, and wherein the determination whether the at least one actuator has detached from the frame comprises a determination whether the at least one actuator has detached from the at least one rod.

Example 13. The system of any example herein, particularly example 12, wherein each of the at least one rod is threaded.

Example 14. The system of any example herein, particularly example 12 or 13, wherein each of the proximal member and distal member of each of the at least one axially extending post comprise an inner bore, the respective one of the at least one rod extending through the inner bores of the proximal member and distal member.

Example 15. The system of any example herein, particularly any one of examples 12 to 14, wherein the distance between the at least one mechanical wave sensor and each of the at least one rod is less than 20 centimeters.

Example 16. The system of any example herein, particularly example 15, wherein the distance between the at least one mechanical wave sensor and each of the at least one rod is less than 10 centimeters.

Example 17. The system of any example herein, particularly example 16, wherein the distance between the at least one mechanical wave sensor and each of the at least one rod is less than 5 centimeters.

Example 18. The system of any example herein, particularly any one of examples 12 to 17, wherein each of the at least one actuator comprises: at least one elongated member; and a movable sleeve configured to compress the pair of elongated members such that the compressed at least one elongated member is fastened to a respective one of the at least one rod, wherein when the movable sleeve is moved away from the at least one elongated member, the at least one elongated member detaches from the respective rod, the determination whether the at least one actuator has detached from the at least one rod comprising a determination whether the at least one elongated member has detached from the respective rod.

Example 19. The system of any example herein, particularly example 18, wherein the at least one elongated member comprises a pair of elongated members.

Example 20. The system of any example herein, particularly example 18 or 19, wherein the at least one elongated member extends distally from the at least one actuator.

Example 21. The system of any example herein, particularly any one of examples 18 to 20, wherein the distal end of the at least one of the pair of elongate members comprises at least one protrusion.

Example 22. The system of any example herein, particularly any one of examples 18 to 20, wherein the distal end of the at least one of the pair of elongate members comprises at least one tooth.

Example 23. The system of any example herein, particularly any one of examples 12 to 22, wherein the at least one axially extending post comprises a plurality of axially extending posts and the at least one rod comprises a plurality of rods, wherein the at least one actuator comprises a plurality of actuators, the rotation of each of the plurality of rods based at least in part on a respective one of the plurality of actuators, and wherein the determination whether the at least one actuator has detached from the frame comprises a determination whether all of the plurality of actuators have detached from the frame.

Example 24. The system of any example herein, particularly any one of examples 11 to 23, wherein the determination whether the actuator has detached from the frame is based at least in part on detecting whether one or more predetermined mechanical wave signals are present in the output of the at least one mechanical wave sensor.

Example 25. The system of any example herein, particularly example 24, wherein the determination whether the plurality of actuators have detached from the frame is based at least in part on detecting a plurality of predetermined mechanical wave signals, and wherein, based at least in part on the output of the mechanical wave sensor, the detection module is further configured to: identify which of the plurality of actuators have detached from the frame; and output an indication of the identified actuators.

Example 26. The system of any example herein, particularly example 25, wherein the at least one mechanical wave sensor comprises a microphone array.

Example 27. The system of any example herein, particularly example 25 or 26, wherein the identification of which of the plurality of actuators have detached from the frame comprises an identification of the time difference between the detection of the predetermined mechanical wave signals.

Example 28. The system of any example herein, particularly any one of examples 11 to 27, wherein the detection module is further configured to: based at least in part on the received output, determine whether the at least one actuator has broken; and based at least in part on the determination, output an indication of whether the at least one actuator has broken.

Example 29. The system of any example herein, particularly example 28, wherein the determination whether the at least one actuator has broken is based at least in part on detecting a respective predetermined sound withing the output of the at least one mechanical wave sensor.

Example 30. The system of any example herein, particularly any one of examples 1 to 29, wherein the at least one mechanical wave sensor is attached to the at least one actuator.

Example 31. The system of any example herein, particularly any one of examples 1 to 29, wherein the at least one mechanical wave sensor is attached to the frame.

Example 32. The system of any example herein, particularly any one of examples 1 to 29, further comprising a delivery shaft, the at least one actuator supported by the delivery shaft, wherein the at least one mechanical wave sensor is attached to the delivery shaft.

Example 33. The system of any example herein, particularly any one of examples 1 to 29, further comprising a nosecone shaft extending through the frame, wherein the at least one mechanical wave sensor is attached to the nosecone shaft.

Example 34. The system of any example herein, particularly any one of examples 1 to 29, further comprising: a handle; a sensor shaft extending from the handle, the mechanical wave sensor secured to the sensor shaft; and a delivery shaft coupled to the handle, the at least one actuator and the sensor shaft supported by the delivery shaft, wherein the at least one mechanical wave sensor extends distally from the delivery shaft.

Example 35. The system of any example herein, particularly any one of examples 1 to 29, further comprising: a handle; a sensor shaft, the at least one mechanical wave sensor secured to the sensor shaft; and a delivery shaft coupled to the handle, the at least one actuator supported by the delivery shaft and the sensor shaft not supported by the delivery shaft.

Example 36. The system of any example herein, particularly any one of examples 1 to 29, further comprising: a handle; a sensor shaft, the at least one mechanical wave sensor secured to the sensor shaft; and a delivery shaft coupled to the handle, the at least one actuator supported by the delivery shaft and the sensor shaft not supported by the delivery shaft, wherein the at least one mechanical wave sensor extends distally to the delivery shaft.

Example 37. The system of any example herein, particularly any one of examples 1 to 29, wherein the at least one mechanical wave sensor is attachable to the skin of a patient.

Example 38. The system of any example herein, particularly example 37, further comprising a sensor housing, the at least one mechanical wave sensor attached to the sensor housing, wherein the sensor housing is attachable to the skin of the patient.

Example 39. The system of any example herein, particularly any one of examples 11 to 38, further comprising an indicator configured to display the indication of whether the at least one actuator has detached from the frame.

Example 40. The system of any example herein, particularly example 39, further comprising a case configured to secure the prosthetic valve therewithin, wherein the case comprises a window aligned with the indicator.

Example 41. The system of any example herein, particularly example 39, further comprising a case configured to secure the prosthetic valve therewithin, wherein the indicator is provided on an external surface of the case.

Example 42. The system of any example herein, particularly example 39, further comprising a case configured to secure the prosthetic valve therewithin, wherein the case is transparent.

Example 43. A prosthetic valve actuator detachment detection method, the method comprising: sensing mechanical waves in originating from at least one actuator secured to a frame of the prosthetic valve movable by the at least one actuator between a radially compressed configuration and a radially expanded configuration; and outputting an indication of the sensed mechanical waves.

Example 44. The method of any example herein, particularly example 43, wherein the mechanical waves are sensed by at least one mechanical wave sensor.

Example 45. The method of any example herein, particularly example 44, wherein the at least one mechanical wave sensor is a sound sensor.

Example 46. The method of any example herein, particularly example 44, wherein the at least one mechanical wave sensor comprises a plurality of mechanical wave sensors.

Example 47. The method of any example herein, particularly example 46, wherein the plurality of mechanical wave sensors comprises a plurality of sound sensors.

Example 48. The method of any example herein, particularly example 46, wherein the plurality of mechanical wave sensors comprises a plurality of vibration sensors.

Example 49. The method of any example herein, particularly example 46, wherein the plurality of mechanical wave sensors comprises at least one sound sensor and at least one vibration sensor.

Example 50. The method of any example herein, particularly example 44, wherein the at least one mechanical wave sensor is a vibration sensor.

Example 51. The method of any example herein, particularly example 44, wherein the at least one mechanical wave sensor comprises a microphone.

Example 52. The method of any example herein, particularly example 51, wherein the microphone is selected from a piezoelectric, a piezoresistive, and a capacitive-type microphone.

Example 53. The method of any example herein, particularly example 51, wherein the microphone is a MEMS microphone.

Example 54. The method of any example herein, particularly any one of examples 44 to 53, further comprising: receiving an output of the at least one mechanical wave sensor; based at least in part on the received output, determining whether the at least one actuator has detached from the frame; and based at least in part on the determination, outputting an indication of whether the at least one actuator has detached from the frame.

Example 55. The method of any example herein, particularly example 54, wherein the frame comprises: at least one axially extending post, each of the at least one axially extending post comprising a proximal member and a distal member that are axially movable relative to one another to permit the frame to radially expand and/or compress, at least one rod, each associated with a respective one of the at least one axially extending post and rotatably coupled to the proximal member and distal member thereof, wherein a rotation of the at least one rod radially expands and/or radially compresses the frame, the rotation of the at least one rod based at least in part on the at least one actuator, and wherein the determination whether the at least one actuator has detached from the frame comprises a determination whether the at least one actuator has detached from the at least one rod.

Example 56. The method of any example herein, particularly example 55, wherein each of the at least one rod is threaded.

Example 57. The method of any example herein, particularly example 55 or 56, wherein each of the proximal member and distal member of each of the at least one axially extending post comprise an inner bore, the respective one of the at least one rod extending through the inner bores of the proximal member and distal member.

Example 58. The method of any example herein, particularly any one of examples 55 to 57, wherein the distance between the at least one mechanical wave sensor and each of the at least one rod is less than 20 centimeters.

Example 59. The method of any example herein, particularly example 58, wherein the distance between the at least one mechanical wave sensor and each of the at least one rod is less than 10 centimeters.

Example 60. The method of any example herein, particularly example 59, wherein the distance between the at least one mechanical wave sensor and each of the at least one rod is less than 5 centimeters.

Example 61. The method of any example herein, particularly any one of examples 55 to 60, wherein each of the at least one actuator comprises: at least one elongated member; and a movable sleeve configured to compress the pair of elongated members such that the compressed at least one elongated member is fastened to a respective one of the at least one rod, wherein when the movable sleeve is moved away from the at least one elongated member, the at least one elongated member detaches from the respective rod, the determination whether the at least one actuator has detached from the at least one rod comprising a determination whether the at least one elongated member has detached from the respective rod.

Example 62. The method of any example herein, particularly example 61, wherein the at least one elongated member comprises a pair of elongated members.

Example 63. The method of any example herein, particularly example 61 or 62, wherein the at least one elongated member extends distally from the at least one actuator.

Example 64. The method of any example herein, particularly any one of examples 61 to 63, wherein the distal end of the at least one of the pair of elongate members comprises at least one protrusion.

Example 65. The method of any example herein, particularly any one of examples 61 to 63, wherein the distal end of the at least one of the pair of elongate members comprises at least one tooth.

Example 66. The method of any example herein, particularly any one of examples 55 to 65, wherein the at least one axially extending post comprises a plurality of axially extending posts and the at least one rod comprises a plurality of rods, wherein the at least one actuator comprises a plurality of actuators, the rotation of each of the plurality of rods based at least in part on a respective one of the plurality of actuators, and wherein the determination whether the at least one actuator has detached from the frame comprises a determination whether all of the plurality of actuators have detached from the frame.

Example 67. The method of any example herein, particularly any one of examples 54 to 66, wherein the determination whether the actuator has detached from the frame is based at least in part on detecting whether one or more predetermined mechanical wave signals are present in the output of the at least one mechanical wave sensor.

Example 68. The method of any example herein, particularly example 67, wherein the determination whether the plurality of actuators have detached from the frame is based at least in part on detecting a plurality of predetermined mechanical wave signals, and wherein, based at least in part on the output of the mechanical wave sensor, the detection module is further configured to: identify which of the plurality of actuators have detached from the frame; and output an indication of the identified actuators.

Example 69. The method of any example herein, particularly example 68, wherein the at least one mechanical wave sensor comprises a microphone array.

Example 70. The method of any example herein, particularly example 68 or 69, wherein the identification of which of the plurality of actuators have detached from the frame comprises an identification of the time difference between the detection of the predetermined mechanical wave signals.

Example 71. The method of any example herein, particularly any one of examples 54 to 70, wherein the detection module is further configured to: based at least in part on the received output, determine whether the at least one actuator has broken; and based at least in part on the determination, output an indication of whether the at least one actuator has broken.

Example 72. The method of any example herein, particularly example 71, wherein the determination whether the at least one actuator has broken is based at least in part on detecting a respective predetermined sound withing the output of the at least one mechanical wave sensor.

Example 73. The method of any example herein, particularly any one of examples 44 to 72, wherein the at least one mechanical wave sensor is attached to the at least one actuator.

Example 74. The method of any example herein, particularly any one of examples 44 to 72, wherein the at least one mechanical wave sensor is attached to the frame.

Example 75. The method of any example herein, particularly any one of examples 44 to 72, wherein the at least one actuator is supported by a delivery shaft, and wherein the at least one mechanical wave sensor is attached to the delivery shaft.

Example 76. The method of any example herein, particularly any one of examples 44 to 72, wherein the at least one mechanical wave sensor is attached to the nosecone shaft, the nosecone shaft extending through the frame.

Example 77. The method of any example herein, particularly any one of examples 44 to 72, wherein the at least one mechanical wave sensor extends distally from a delivery shaft and is secured to a sensor shaft, the delivery shaft coupled to a handle, and wherein the at least one actuator and the sensor shaft are supported by the delivery shaft.

Example 78. The method of any example herein, particularly any one of examples 44 to 72, wherein the at least one mechanical wave sensor extends distally from a delivery shaft and is secured to a sensor shaft, the delivery shaft coupled to a handle, and wherein the at least one actuator is supported by the delivery shaft and the sensor shaft is not supported by the delivery shaft.

Example 79. The method of any example herein, particularly any one of examples 44 to 72, wherein the at least one mechanical wave sensor extends distally to a delivery shaft and is secured to a sensor shaft, the delivery shaft coupled to a handle, and wherein the at least one actuator is supported by the delivery shaft and the sensor shaft is not supported by the delivery shaft.

Example 80. The method of any example herein, particularly any one of examples 44 to 72, wherein the at least one mechanical wave sensor is attachable to the skin of a patient.

Example 81. The method of any example herein, particularly example 80, wherein the at least one mechanical wave sensor is attached to a sensor housing, wherein the sensor housing is attachable to the skin of the patient.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve actuator detachment detection system, comprising:
at least one actuator;
a frame of the prosthetic valve movable by the at least one actuator between a radially compressed configuration and a radially expanded configuration; and
at least one mechanical wave sensor.

2. The system of claim 1, wherein the at least one mechanical wave sensor is a sound sensor.

3. The system of claim 1, wherein the at least one mechanical wave sensor is a vibration sensor.

4. The system of claim 1, further comprising a detection module configured to:
receive an output of the at least one mechanical wave sensor;
based at least in part on the received output, determine whether the at least one actuator has detached from the frame; and
based at least in part on the determination, output an indication of whether the at least one actuator has detached from the frame.

5. The system of claim 4, wherein the frame comprises: at least one axially extending post, each of the at least one axially extending post comprising a proximal member and a distal member that are axially movable relative to one another to permit the frame to radially expand and/or compress,
at least one rod, each associated with a respective one of the at least one axially extending post and rotatably coupled to the proximal member and distal member thereof,
wherein a rotation of the at least one rod radially expands and/or radially compresses the frame, the rotation of the at least one rod based at least in part on the at least one actuator, and
wherein the determination whether the at least one actuator has detached from the frame comprises a determination whether the at least one actuator has detached from the at least one rod.

6. The system of claim 5, wherein each of the proximal member and the distal member of each of the at least one axially extending post comprises an inner bore, the respective one of the at least one rod extending through the inner bores of the proximal member and distal member.

7. The system of claim 5, wherein each of the at least one actuator comprises:
a pair of elongated members; and
a movable sleeve configured to compress the pair of elongated members such that the compressed pair of elongated members are fastened to a respective one of the at least one rod,
wherein when the movable sleeve is moved away from the pair of elongated members the pair of elongated members detach from the respective rod, the determination whether the at least one actuator has detached from the at least one rod comprising a determination whether the pair of elongated members have detached from the respective rod.

8. The system of claim 5, wherein the at least one axially extending post comprises a plurality of axially extending posts and the at least one rod comprises a plurality of rods,
wherein the at least one actuator comprises a plurality of actuators, the rotation of each of the plurality of rods based at least in part on a respective one of the plurality of actuators, and
wherein the determination whether the at least one actuator has detached from the frame comprises a determination whether all of the plurality of actuators have detached from the frame.

9. The system of claim 4, wherein the determination whether the actuator has detached from the frame is based at least in part on detecting whether one or more predetermined mechanical wave signals are present in the output of the at least one mechanical wave sensor.

10. The system of claim 8, wherein the determination whether the plurality of actuators have detached from the frame is based at least in part on detecting a plurality of predetermined mechanical wave signals,
wherein the at least one mechanical wave sensor comprises a microphone array, and
wherein, based at least in part on the output of the mechanical wave sensor, the detection module is further configured to:
identify which of the plurality of actuators have detached from the frame; and
output an indication of the identified actuators.

11. The system of claim 4, wherein the detection module is further configured to:
based at least in part on the received output, determine whether the at least one actuator has broken; and
based at least in part on the determination, output an indication of whether the at least one actuator has broken.

12. The system of claim 1, wherein the at least one mechanical wave sensor is attached to the at least one actuator.

13. The system of claim 1, wherein the at least one mechanical wave sensor is attached to the frame.

14. The system of claim 1, further comprising a delivery shaft, the at least one actuator supported by the delivery shaft,
wherein the at least one mechanical wave sensor is attached to the delivery shaft.

15. The system of claim 1, further comprising a nosecone shaft extending through the frame,
wherein the at least one mechanical wave sensor is attached to the nosecone shaft.

16. The system of claim 1, further comprising:
a handle;
a sensor shaft extending from the handle, the mechanical wave sensor secured to the sensor shaft; and
a delivery shaft coupled to the handle, the at least one actuator and the sensor shaft supported by the delivery shaft,
wherein the at least one mechanical wave sensor extends distally from the delivery shaft.

17. The system of claim 1, further comprising:
a handle;
a sensor shaft, the at least one mechanical wave sensor secured to the sensor shaft; and
a delivery shaft coupled to the handle, the at least one actuator supported by the delivery shaft and the sensor shaft not supported by the delivery shaft.

18. The system of claim 1, wherein the at least one mechanical wave sensor is attachable to the skin of a patient.

19. The system of claim 18, further comprising a sensor housing, the at least one mechanical wave sensor attached to the sensor housing,
wherein the sensor housing is attachable to the skin of the patient.
